(19) United States
(12) Patent Application Publication
(10) Pub. No.: US 2022/0054046 A1
(43) Pub. Date: Feb. 24, 2022

(54) ASSESSING PATIENT OUT-OF-BED AND OUT-OF-CHAIR ACTIVITIES USING EMBEDDED INFRARED THERMAL CAMERAS

(71) Applicant: Envision Analytics, Inc., Benton, AR (US)

(72) Inventors: Christophe Bobda, Newberry, FL (US); Lenny Baledge, Prairie Grove, AR (US); Lance Porter, West Fork, AR (US); Rudy Timmerman, Prairie Grove, AR (US)

(21) Appl. No.: 17/409,259

(22) Filed: Aug. 23, 2021

Related U.S. Application Data
(60) Provisional application No. 62/706,531, filed on Aug. 23, 2020.

(51) Int. Cl. A61B 5/11; A61B 5/00

(52) U.S. Cl. CPC A61B 5/1115; A61B 5/0077; A61B 5/746; A61B 5/1128

(57) ABSTRACT

A patient monitoring system. The patient monitoring system comprises a camera and a processing unit in communication with the camera. The processing unit is configured to determine a safe zone around the patient based at least in part on an analysis of one or more initial images received from the camera. The processing unit is further configured to determine whether the patient has exited the safe zone based at least in part on an analysis of one or more subsequent images received from the camera. The processing unit is also configured to trigger an alarm in response to determining that the patient has exited the safe zone.

### CROSS-REFERENCE TO RELATED APPLICATION

The present application claims the benefit of the filing date of, and priority to, U.S. Application No. 62/706,531, filed Aug. 23, 2020, the entire disclosure of which is hereby incorporated herein by reference.

### BACKGROUND

Falls suffered by the elderly are a growing concern as people age and are a common complaint to accident and emergency departments. Falls are a complex geriatric syndrome with various consequences ranging from mortality, morbidity, reduced functioning, and premature nursing home admissions. Around 40% of people aged 65 years and older fall annually, a rate which increases above 50% with advanced age and among people who live in residential care facilities or nursing homes. About 20% of those who fall need medical attention, 5% result in bone fractures and other serious injuries, including severe head injuries, joint distortions and dislocations. Soft-tissue bruises, contusions, and lacerations occur in 5 to 10% of cases. These percentages can be more than doubled for women aged 75 years or older. The cost to the healthcare system resulting from falls and fall-related injuries was $34 B in 2013 and $50 B in 2015. This represents a growth rate of more than 30% over 2 years, a pace that will result in costs of more than $100 B in the next 10 years. This high economic impact does not assess resulting individual morbidity (disability, dependence, depression, unemployment, inactivity).

Methods to address falls can be classified in three categories. Fall prevention, early fall detection, and prevention of injuries resulting from falls. While early fall detection and limitation off fall related injuries can help, they are only helpful when falls have already happened. Prevention and intervention measures that seek to eliminate falls are highly desirable.

Published work on fall prevention addresses off-line patient health history, medication and environmental assessment. Hospitals and care facilities today use bed mats, which incorporate pressure sensors to detect patients leaving their beds and alerting the caregivers. The lack of a global view of the scene leads to a high number of false positives with frequent and time-consuming unnecessary visits to patients' rooms. Surveillance cameras can provide a global view of the scene but require a human behind a screen for real-time assessment. Also, image streaming to a remote station creates privacy concerns and limits the adoption of this technology.

### SUMMARY

In one exemplary aspect, a patient monitoring system is disclosed. The patient monitoring system comprises a camera and a processing unit in communication with the camera. The processing unit is configured to determine, based at least in part on an analysis of one or more initial images received from the camera, a safe zone around the patient. The processing unit is further configured to determine, based at least in part on an analysis of one or more subsequent images received from the camera, whether the patient has exited the safe zone. The processing unit is also configured to trigger an alarm in response to determining that the patient has exited the safe zone. The processing unit is further configured to calculate a shift metric based at least in part on patient movement detected in one or more of the subsequent image and to adjust the safe zone based at least in part on the shift metric. Determining whether the patient has exited the safe zone also comprises determining an N number of safe zone perimeter pixels touched by an image object representative of the patient and determining whether the image object touches greater than N number of pixel layers outside the safe zone. The processing unit is further configured to calculate a cloak metric based at least in part on a level of patient occlusion detected by the processing unit and determine whether the patient has exited the safe zone based at least in part on the cloak metric. The processing unit is further configured to calculate a fidget index based at least in part on an amount of patient movement detected in the subsequent images. The processing unit is further configured to trigger an alarm when the fidget index exceeds a threshold. The camera also may be a thermal camera. Determining whether the patient has exited the safe zone also comprises tracking a silhouette of the patient's head and shoulders. The processing unit is also configured to determine the safe zone around the patient in response to detecting an enable gesture in one or more of the initial images.

In another exemplary aspect, a patient monitoring method is disclosed. The patient monitoring method comprises determining, by a processing unit in communication with a camera, a safe zone around the patient based at least in part on an analysis of one or more initial images received from the camera. The patient monitoring method further comprises determining, by the processing unit, whether the patient has exited the safe zone based at least in part on an analysis of one or more subsequent images received from the camera. The patient monitoring method also comprises triggering, by the processing unit, an alarm in response to determining that the patient has exited the safe zone. The patient monitoring method further comprises calculating, by the processing unit, a shift metric based at least in part on patient movement detected in one or more of the subsequent images and adjusting, by the processing unit, the safe zone based at least in part on the shift metric. Determining whether the patient has exited the safe zone comprises determining an N number of safe zone perimeter pixels touched by an image object representative of the patient and determining whether the image object touches greater than N number of pixel layers outside the safe zone. The patient monitoring method includes calculating, by the processing unit, a cloak metric based at least in part on a level of patient occlusion detected by the processing unit and determining, by the processing unit, whether the patient has exited the safe zone based at least in part on the cloak metric. The patient monitoring method further comprises calculating, by the processing unit, a fidget index based at least in part on an amount of patient movement detected in the subsequent images. The patient monitoring method further comprises triggering, by the processing unit, an alarm when the fidget index exceeds a threshold.

In yet another exemplary aspect, another patient monitoring system is disclosed. The patient monitoring system comprises a thermal camera and a processing unit in communication with the thermal camera. The processing unit is configured to determine, based at least in part on an analysis of one or more initial thermal images received from the thermal camera, a safe zone around the patient. The processing unit is further configured to determine, based at least in part on an analysis of one or more subsequent thermal images received from the thermal camera, whether the patient has exited the safe zone. The processing unit is also configured to trigger an alarm in response to determining that the patient has exited the safe zone. Determining whether the patient has exited the safe zone comprises determining whether hot pixels completely fill a column of pixels from top to bottom of a thermal image. Determining whether the patient has exited the safe zone comprises determining whether one or more pixels have changed temperature in two or more consecutive thermal images received from the thermal camera. Determining whether the patient has exited the safe zone comprises comparing one or more initial thermal images with one or more subsequent thermal images taken a pre-determined amount of time after the one or more initial thermal images used in the comparison were taken. Determining the safe zone comprises determining a density score of hot pixels to total area within a boundary. Determining the safe zone comprises at least one of determining a ratio of height-to-width or determining a distance to an edge of a thermal image.

### BRIEF DESCRIPTION OF THE DRAWINGS

Reference will now be made to the accompanying drawings, of which:

FIG. 1 is a diagrammatic view of a solution architecture, according to aspects of the present disclosure.

FIG. 2 is diagram of a camera platform, according to aspects of the present disclosure.

FIG. 3A is a diagram illustrating a position of a subject relative to a safe zone, according to aspects of the present disclosure.

FIG. 3B is a diagram illustrating a position of a subject relative to a safe zone, according to aspects of the present disclosure.

FIG. 3C is a diagram illustrating a position of a subject relative to a safe zone, according to aspects of the present disclosure.

FIG. 3D is a diagram illustrating a position of a subject relative to a safe zone, according to aspects of the present disclosure.

FIG. 4A is a diagram of a thermal image, according to aspects of the present disclosure.

FIG. 4B is a diagram of a thermal image, according to aspects of the present disclosure.

FIG. 5 is a schematic diagram of a processing unit, according to aspects of the present disclosure.

FIG. 6 is a flow diagram of a method, according to aspects of the present disclosure.

### DETAILED DESCRIPTION

To overcome the deficiencies of previous solutions, the current disclosure presents a contact-less, multi-sensor smart camera solution along with a machine learning inference model for assessment of patient surroundings and notification of imminent fall risk. With a smart notification system, the current disclosure empowers caregivers with efficient tools for fast and sound decision making that limit disruptions while providing care to other patients.

The descriptions herein are provided for exemplary purposes and should not be considered to limit the scope of the disclosure. Certain features may be added, removed, or modified without departing from the spirit of the claimed subject matter. For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

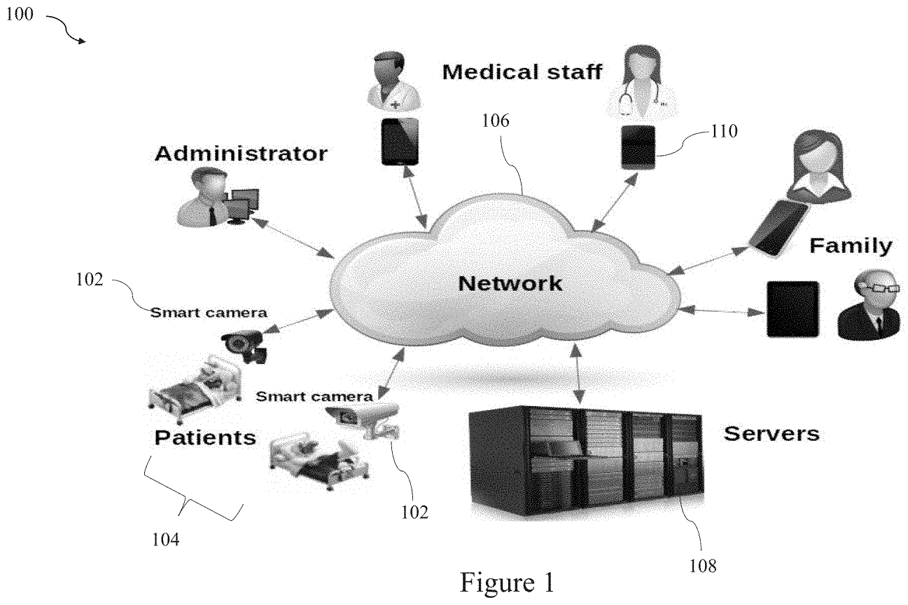

Turning now to FIG. 1, a system 100 is described. In an embodiment, the system 100 comprises cameras 102, servers 108, and user devices 110 connected via a network 106. Network 106 may comprise a local area network (LAN), enterprise network, wide area network (WAN), virtual private network (VPN), personal area network (PAN), campus network, or any combination thereof. User devices 110 may be portable, e.g., a mobile phone or tablet, or may be stationary, e.g., a desktop computer. By way of further example, it is specifically contemplated that user devices 110 may comprise personal digital assistants (PDA), laptop computers, digital whiteboards, television sets, pagers, notebook computers, or any combination thereof. Servers 108 may be located on premises with cameras 102, e.g., at a hospital or care facility, or may be located remotely. Though a plurality of cameras 102, servers 108, and user devices 110 are illustrated in FIG. 1, it should be understood that in some embodiments a single camera 102, server 108, or user device 110 may be used. One or more aspects of system 100 may be located in a hospital, care facility, private home, or other facility. In that regard, one or more patients 104 may be looked after by a caregiver, e.g., by medical staff or family shown in FIG. 1. The patient environment could be a single room, a home, or a part of a home or facility.

In system 100, a camera 102 views the environment around a patient 104 and may be configured to receive input from caregivers. The input may comprise configuration parameters stating how camera 102 should behave and what type of activities should be tracked. Upon detecting predefined actions and events in regions of interest, a camera 102 can trigger alarms. Those alarms are forwarded to servers 108, which can be hosted on the Internet or a local network and may be accessible through webservices. Notifications received by a server 108 from a camera 102 are forwarded to the caregivers and displayed on their mobile devices 110. Alarm notifications may be forwarded to all caregivers or to a subset of caregivers. The subset of caregivers may be determined based on a rule set defining under which conditions a particular caregiver is to be notified. For example, the rule set may specify that medical staff are only to receive such notifications when they are clocked-in or otherwise recognized as being at work. Caregivers are registered in the system by the system administrators who ensure proper functioning of the whole infrastructure. In some embodiments, the processing of information may take place within camera 102, thus advantageously preserving the patient's privacy.

Camera 102 may comprise a multi-sensor smart camera platform and may be alternatively referred to as a smart camera, smart camera device, or simply device. Camera 102 may use two different image sources to ensure that all events are accurately detected. In that regard, camera 102 uses one or more sensors to capture the scene as shown in FIG. 1. In an embodiment, the main sensor is a long-wave infrared (LWIR) thermal sensor used to accurately detect people in the scene based on their body temperature. Existing person detection and tracking algorithms that are based on color sensors still have a relatively high rate of false positives for the goal of this project. Using a thermal sensor as the main sensor advantageously allows detection to more closely approach an accuracy of 100%. Even so, a color sensor may be used in addition to the thermal sensor to improve detection and tracking quality. Camera 102 may be connected to a communication module (wired or wireless) through which it can connect to network 106, which may be an internal or external network, to receive inputs such as configuration data and through which it can broadcast or otherwise transmit notifications. In some embodiments, multiple cameras 102 may observe a single scene such a patient room in order improve the accuracy of event detection. In such embodiments, one of the cameras 102 may comprise a thermal sensor while the other comprises a color sensor.

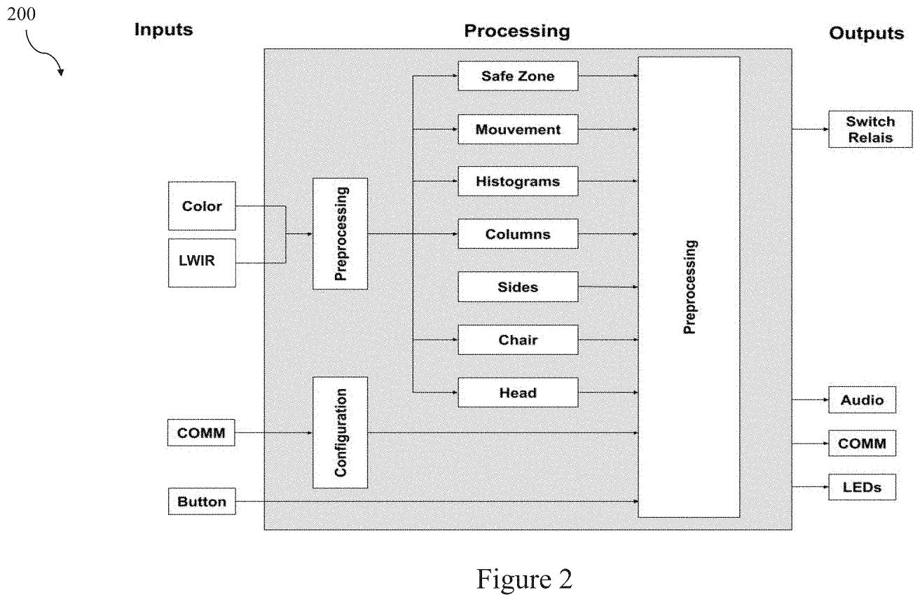

Turning now to FIG. 2, a processing module 200 is described. Processing module 200 may be included in camera 102 or within another device. The processing module 200 may comprise or be in communication with a decision manager. The decision manager may be part of a camera, e.g., camera 102, may be part of a server, e.g., server 108, or may be part of some other device. The decision manager maintains various states of the system, e.g., enabled, visitor present, chair scenario detected, etc. These states and various combinations of metrics received from other modules, e.g., processing module 200, are then used to determine when and if an alarm is issued. Alarms are generated in response to various combinations of factors. These combinations were derived using a set of several hundred pre-recorded videos of thermal images made using LWIR hardware, sample rates, and pre-processing as in normal operation. One alarm might address one patient exit behavior, for example, leaving the frame completely through the left/right side, while another addresses another patient exit behavior, for example, leaving the nearby region but remaining in the frame. These alarms may be logically OR'd together.

As shown in FIG. 2, the processing module 200 can produce various output signals depending on the configuration. Outputs can include switch relays, audio signals, COMM signals, and LED activation. Simple status information may be conveyed with one or more LEDs, including, for example, bi-color LEDs. The LEDs indicate whether the system is disabled (e.g., OFF), enabled (e.g., GREEN), or alarming (e.g., blinking RED). Such LED indications may be outputs of the processing module 200 as described above. The primary alarm output may be a simple relay. This would integrate well into many existing hospital's infrastructure by allowing the replacement of the output of a bed mat monitor. More structured notifications may be sent to an advanced notification system through a communication network, e.g., network 106 described with reference to FIG. 1.

Regarding inputs, processing module 200 may be in communication with a button used by the caregiver to enable or disable a camera, e.g., camera 102. An on-board audio transducer may provide user feedback for button presses and can optionally be used for audible alarm signals. Many patient advocates consider audible alarms near the patient to be a form of restraint, therefore the audio alarm may be disabled by default but can be enabled or otherwise reconfigured at the discretion of the care facility. As shown in FIG. 2, other inputs include color signals from a color sensor, LWIR signals from a thermal sensor, and COMM signals.

Regarding the processing aspect shown in FIG. 2, information from the thermal and color-image sensor are processed in the processing module 200. The processing module 200 can comprise a microprocessor running bare metal or running an operating system, a VLSI chip, an FPGA, or any combination of these components, on a single chip, a multi-chip module, or a printed circuit board. The thermal sensor may serve as the primary sensor for identifying people, including patients, caregivers, and visitors. Processing of information from the sensors may take place in several stages, including pre-processing, calibration, events detection, and notification.

In one aspect, the pre-processing converts data from the sensors to a format with reduced amount of data without loss of information. This may be referred to in some contexts as quantization. The processing module 200 may comprise a pre-processing module as shown in FIG. 2. The pre-processing module may accept frames of 14-bit pixels in 80×60 format and convert the pixels to a more convenient frame of 8-bit pixels. The LWIR is not strictly limited to imaging humans and has a wider dynamic range than the minimum adequacy for the application at hand. The pre-processing algorithm tracks the average histogram of the entire frame and finds the offset and scalar that best maps this to 8-bits.

In several parts of the processing, a simple infinite impulse response (IIR) filter may be used to average data, including individual pixels. An exemplary form is:

y[n]=y[n−1]+(x[n]−y[n−1])*u

where u may very often be implemented with a shift, limiting it to powers of two. This can be realized entirely in hardware or on a fixed-point processor with minimal resources and advantageously gives a wide range of performance control using only the one parameter, u. One may avoid spending undue time analyzing or optimizing these filters by simply sweeping the value u quickly until the desired effect is achieved. Filters may be referred to by the value of u or the time constant which is approximately (1/u) in frames.

Before detection and tracking, the device should be calibrated to ensure that the region of tracking is captured. This operation may be done in a very simple way upon physical installation of the camera in the patient room. Following the calibration, video streams are analyzed from the input sensors to detect out-of-bed, out-of-chair, and other relevant events such as a patient rolling over or a patient absent in the tracking area.

The purpose of the calibration is to define the region of interest, also called the safe zone or safe region, where the patient is expected to remain. An alarm may be raised when the patient is leaving or has left that area. The detection algorithm should anticipate such actions and notify the caregivers before the action is completed.

At a high-level, the software processes thermal images from the thermal sensor and determines whether the pixels representing the patient are in a safe region of the 2D frame or not. The key to this is determining what constitutes that safe region. Rather than require caregivers to aim precisely or perform a complicated calibration procedure, the safe region is estimated and tracked over time automatically, e.g., by processing module 200.

In an embodiment, the caregiver presses the button resulting in an assumption that the patient is in the frame near the center and is safe. The first few frames, e.g., one or more frames, after the button press are averaged, then a morphological close is performed to create an initial safe zone. These first frames may be referred to as initial images. Over time, the averaging of frames continues, with a slow time constant. For example, a 5-minute time constant may be used. Alternatively, a one minute time constant, two minute time constant, three minute time constant, four minute time constant, six minute time constant, seven minute time constant, eight minute time constant, nine minute time constant, ten minute time constant, a longer than ten minute time constant, or any time constant in between the foregoing values may be used. The time constant can vary depending on the image sensor, the context, or other parameters. The safe zone also automatically adjust to patient shifts in position, covering or uncovering with blankets, etc. A slow time constant, e.g., approximately 5 minutes, ensures the safe zone doesn't track fast enough to allow patients to exit.

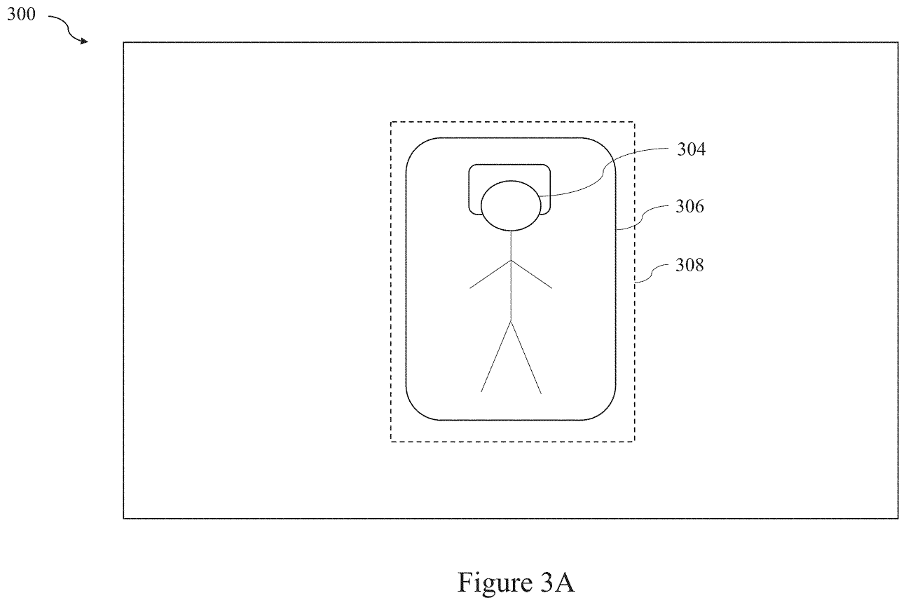

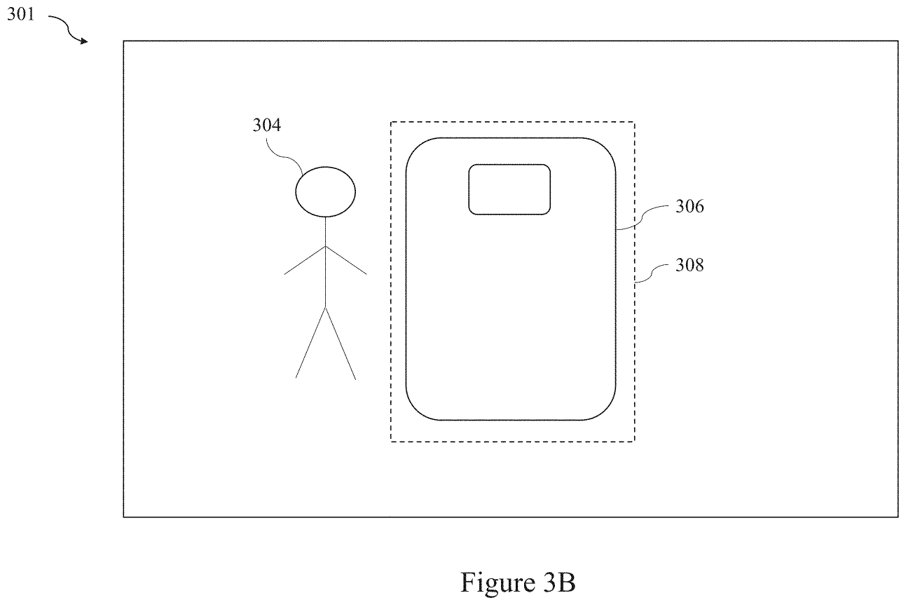

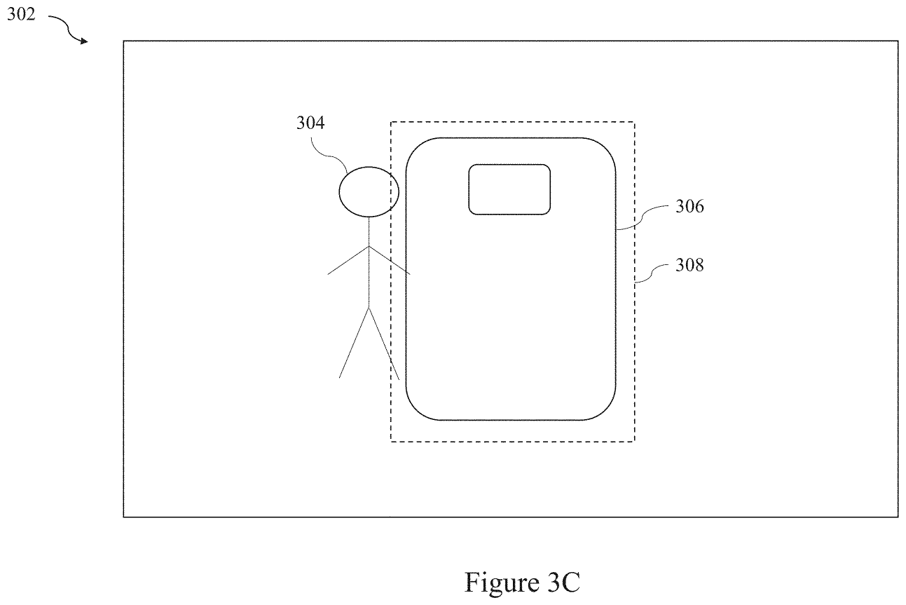

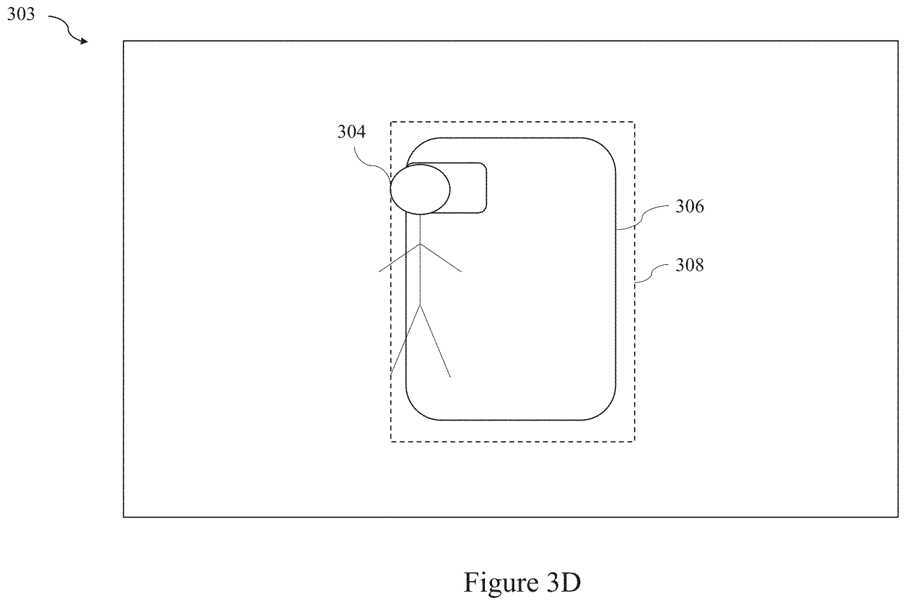

Turning now to FIG. 3A, an image 300 is described. Image 300 may comprise a thermal image from the thermal sensor, a color image from the color sensor, a composite image from multiple thermal sensors, a composite image from multiple color sensors, or a composite image from at least one thermal sensor and at least one color sensor. Image 300 comprises a patient 304 laying on a bed 306 within a safe zone 308. Safe zone 308 may not visibly appear in image 300. Safe zone 308 may be created as described above. In that regard, image 300 may be representative of the patient 304 in an initial position where safe zone 308 is an initial safe zone. Image 300 or portions thereof, e.g., safe zone 308, may serve as a reference to which subsequent images are compared. As the camera captures additional images, e.g., camera 102 capturing thermal images, color images, or both, each image frame is compared to the safe zone 308. Frames compared to the safe zone may be referred to as subsequent images in that they occur after the first frames (initial images) used to create the safe zone 308.

Pixels may be sorted into two new frames—an inside frame and a nearby frame. The inside frame may contain all pixels of the current frame that overlap with safe zone 308. The nearby frame encompasses the inside frame and includes pixels close to safe zone 308. To define which pixels are near safe zone 308, the frame is processed to identify unique objects. Objects are sorted into those that at least partially touch safe zone 308 and those that do not. For example, images 302 and 303 of FIGS. 3C and 3D, respectively, both show patient 304 partially touching safe zone 308. By contrast, image 301 of FIG. 3B shows patient 304 completely outside safe zone 308, which would trigger an alarm.

Not all pixels of an object touching safe zone 308 are considered nearby. A count is made of the number of safe zone perimeter pixels the object touches. If the object touches N perimeter pixels of safe zone 308 then only N layers of pixels outside safe zone 308 are allowed. The result of this is to accelerate the detection of patient 304 leaving. For example, if patient 304 has exited, except for 1 hand reaching back touching the bed (see, for example, FIG. 3C) then only a few pixels of the hand/arm will intersect the safe zone perimeter and only a few pixels will be added to the nearby region. All the other pixels of patient 304 appear outside and can trigger an alarm. Triggering such alarms before patient 304 has completely exited safe zone 308 can advantageously improve response time of caregivers and thereby reduce the likelihood of a fall or other injury.

On the other hand, if patient 304 remains largely within safe zone 308 but has extended some body portion slightly outside the perimeter (see, for example, FIG. 3D) as might occur when patient 304 rolls to one side of bed 306, then the portion of patient 304 that is outside of the safe zone 308 may be deemed acceptably nearby based on the number pixels of patient 304 contacting the perimeter. Allowing for small extensions outside safe zone 308 advantageously reduces the numbers of false alarms sent to caregivers.

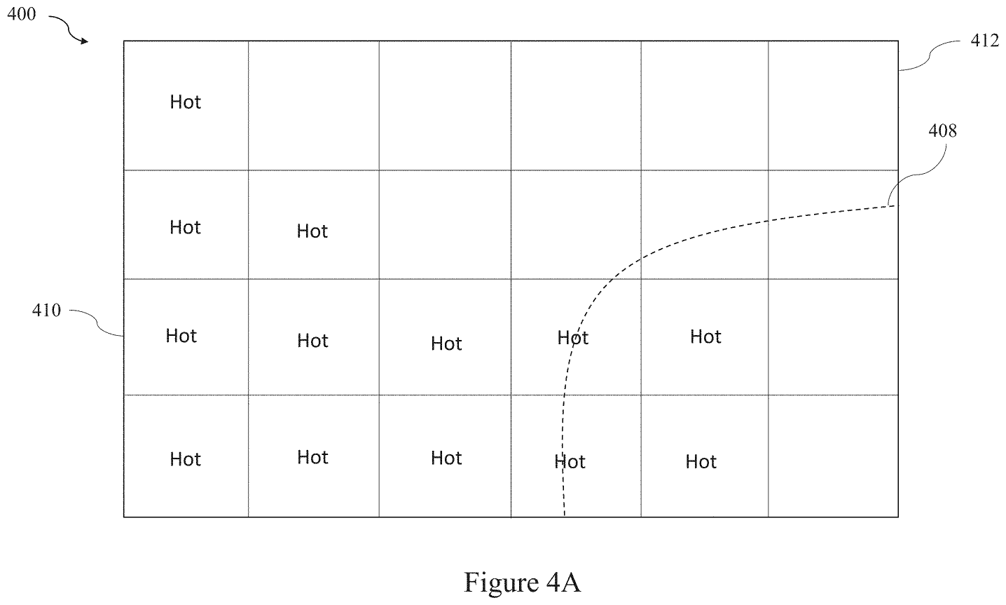

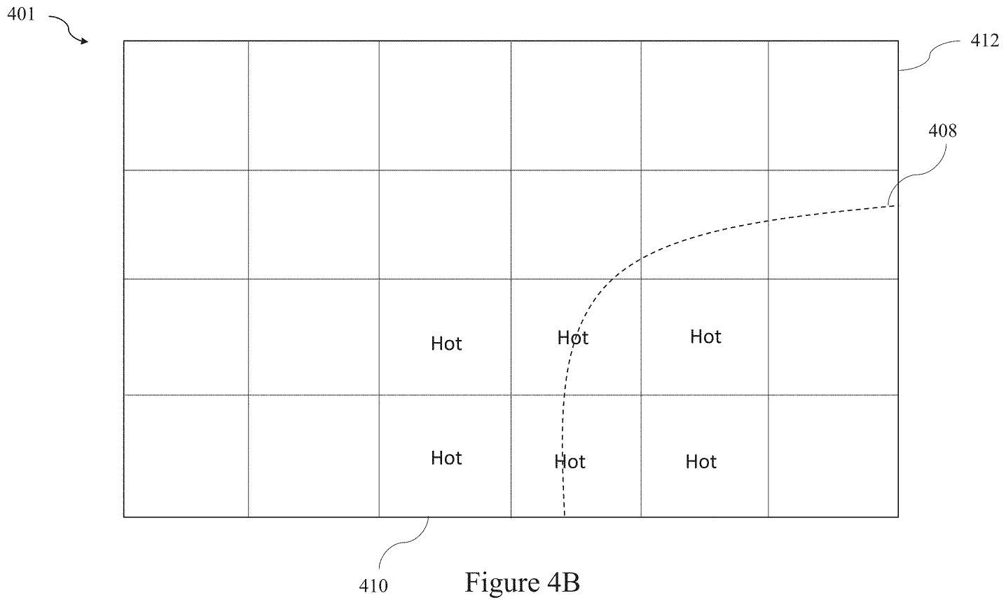

FIGS. 4A and 4B provide thermal images 400 and 401, respectively. These images more clearly show the pixel-level difference between an object being acceptably nearby and an object becoming impermissibly remote. The object may be a patient. In both figures, hot pixels 410 indicate the object while cold pixels 412 indicate the absence of the object. In FIGS. 4A and 4B, only two hot pixels, i.e., pixels representative of the object, contact the perimeter of safe zone 408. Thus, according to the formula above, the object will be permissibly nearby if it extends outside the safe zone 408 by two or fewer layers of pixels. In FIG. 4A, three layers of hot pixels are shown extending past the perimeter of safe zone 408. Therefore, the object may be deemed to be exiting or at risk of exiting the safe zone 408, and an alarm may be triggered. FIG. 4B shows extension outside of only a single layer of pixels, which would result in no alarm. Additionally or alternatively, total number of pixels representative of the object inside safe zone 408 and total number of pixels representative of the object outside safe zone 408 may be used to determine whether to trigger an alarm in some embodiments.

Other metrics may be considered in determining whether a patient has left a safe zone. A count may be made of the number of pixels total in the frame, as well as the total count inside the safe zone and nearby the safe zone. These counts are averaged and used to create two metrics of interest, the shift metric and the cloak metric.
Z=the area of the safe zone itself
S=the average number of hot pixels inside the safe zone
N=the average number of hot pixels nearby (and inside) the safe zone
The shift metric reflects how much the patient has shifted position.

shift=(N−S)/S

The cloak metric reflects the level of occlusion (e.g. blanket covering)

cloak=(Z−S)/S

These values are used by the decision manager for tracking and decision making.

Cameras can also detect of events using as a fusion of knowledge from various submodules. Detection techniques include body tracking, motion detection, head and shoulders tracking, determining the presence or absence of additional people, etc.

Human movement in the thermal images appears as changes in intensity of pixels. However, there are situations where non-human heat sources can change temperature. This is especially true for the residual heat that remains on surfaces (e.g. pillows, blankets, sheets) after the patient moves or exits. The residual heat on surfaces will decay slowly over several seconds to several minutes depending on the surface and total amount of heat involved. To separate this source of heat decay from human movement, look for monotonicity in the changes. A pixel that increases or decreases in two consecutive frames is very likely due to human movement. Non-human heat sources tend to change temperature slowly, typically moving up or down in temperature (example only 1 frame at a time) at least after quantizing to eliminate noise. Some pixels indicating human movement are weighted more heavily than others in analyzing movement. Pixels that are monitored according to where they are in the frame include: inside pixels—pixels inside the safe zone; nearby pixels—pixels in objects substantially in the safe zone; and remote pixels—all other pixels. A count of how many such pixels exist in each region of each frame is made and filtered with u=0.25 to reduce noise. These averaged values are passed to the decision manager.

One movement that may be detected in the thermal images are gestures. Gestures may be supported to generate enable and disable signals. For example, gestures may be used to enable or disable a recording, enable or disable a particular sensor, enable or disable a particular camera, enable or disable connection to a network, enable or disable a functionality of a camera (e.g., audible alarms, tracking functionality, etc.), or any combination thereof. To give a command via gesture, a caregiver's hand/arm may be placed near (6″-18″ away) the sensor so that a large number of pixels of the frame are affected. The interval (6″-18″) may vary depending on the size and type of thermal and image sensor. Example gestures include a movement from bottom-to-top of the frame to trigger an enable, a movement from top-to-bottom to trigger a disable, a movement from side-to-side to scroll through a list of options to be enabled or disabled, or any combination thereof.

To detect these gestures the algorithm maintains an estimate of the background image—those pixels not changing, including a still patient. Each new frame is compared to this background frame. The frame is partitioned into an upper region and a lower region and a count is made of the number of pixels in each region that differ from the background image. Each gesture starts and stops with the background image and progresses from one region to the other in a short time, e.g., 1 sec. Detected events are passed to the decision manager.

Another movement that may be detected is the entrance or exit of visitors or caregivers. As an aid to detection of visitors or caregivers entering the frame, and also as an aid to detection of patient exits, the regions at both the left and right sides of the frame may be monitored. The number of hot pixels in either the left-most 10 pixels or the right-most 10 pixels may be counted. Though 10 pixels is used herein by way of example, it should be understood that other numbers of pixels may likewise be used, e.g., 1, 2, 3, 4, 5, 6, 7, 8, 9, 10, 11, 12, 13, 14, 15, 16, 17, 18, 19, 20, etc. The 10 pixels may be adequate or even optimal for some thermal sensor but the number may vary for other thermal sensors. Next, two separate filters are applied—a fast and a slow filter. The fast filter may have u=0.5 and the slow filter may have u=0.004. The filter parameters can be adjusted according to the image size or other parameters. These data are provided to the decision manager for use with other data. If the fast filter value is significantly greater than the slow filtered value this can indicate an arrival. Similarly, a sudden drop of the fast value below the slow value can indicate an exit of visitor, caregiver, or patient.

To accelerate detection of bed exits when the histograms, described in further detail below, are otherwise slow to respond, changes in vertical columns of pixels can be monitored. When the sensor is positioned close to the bed and a patient exits, the result is often hot pixels fully filling the frame from top-to-bottom. To detect this, start by identifying pixels outside the safe zone. Then include pixels within a pixel bounding box, e.g., a 15×15 bounding box, of identified human movement. The size of the bounding box could be variable according to the size of image size. From this derived frame, create a metric as a weighted percentage of how much of the vertical column is filled. The pixels near the extremities may be weighted more than those near the center. This result is provided to the decision manager for use with other data.

Movement can also be detected using a simple head and shoulders silhouette as a binary template. This pattern can be compared to various locations within the frame. At each location a sum of absolute differences is calculated. The minimum sum and the associated coordinates is found. The sum is normalized and averaged and forms the basis of a head quality metric. If the head quality metric is poor then the template is compared to the region around the top of a bounding box, such that described below. If the head quality metrics are good, then the template is compared around the previous head location. The head quality metric and head location are passed to the decision manager.

In recognition of the fact that patient attempts to exit the bed or chair are often preceded by excessive movement, a fidget index may be calculated. The fidget index may be a windowed sum of movement pixels within, and/or nearby, the safe zone. The sum may be normalized by the size of the safe zone and may have a time window chosen for optimal predictive use, such as 15 seconds. This time is intended by way of non-limiting example and could be longer or shorter in different embodiments. If a patient's fidget index exceeds some given threshold over that time window, then an early warning indication may be sent to caregivers via the wired or wireless link. This early warning advantageously allows caregivers an opportunity to intervene with the patient prior to a safe zone exit thereby reducing the likelihood of a fall or injury.

Each frame may be used to update a continuous estimate of a single bounding box containing hot pixels. The techniques described herein relating to the bounding box may be particularly useful in monitoring a patient in a chair. In some embodiments, the bounding box may comprise a safe zone or a portion of a safe zone. In some embodiments, the bounding box may define a safe zone. The bounding box may be initialized to a fixed set of coordinates encompassing the interior of the image. During each subsequent frame a count is made of the number of pixels in the bounding rectangle and that number is normalized by the total area of that rectangle. This creates a density score for the rectangle.

Candidate rectangles are also considered, where each edge of the rectangle, left, right, top, bottom, is changed by +/−1 pixel. For each such candidate position change, a new density score is calculated. If the density is improved by this candidate score, then a fractional change is added to that edge in that direction. The changes are fractional to reduce noise. In some embodiments, a single frame cannot cause the bounding box to change. The position, dimensions, and density of this bounding box are used to create a metric related to the likelihood that this bounding box contains a patient, e.g., a seated patient. Density may be the primary basis of the metric but adjustments (including penalties or bonuses) may be made based on one or more of: a very small/large ratio of height-to-width, a very large width, a very small width, a very small total area, a very large total area, or a position too close to the left/right side of the frame. The resulting metric and bounding coordinates are passed to the decision manager.

Each inside frame, containing only those pixels inside the safe zone, may be converted into a raw histogram with 16 bins. Each pixel is quantized to 4-bits and that value selects which bin in the histogram is incremented. Raw histograms may be filtered through two sets of filters. The fast histogram is the result of filtering with u=0.5 and the slow histogram is the result of filtering with u=0.002. The purpose of the fast histogram is to remove a small amount of noise. The purpose of the slow histogram is to insert a long time delay in the response, e.g., 512 frames at 8.66 fps is approximately 1 minute. The fast and slow histograms may be normalized and then compared. This effectively allows a comparison between the histogram now and the histogram from a minute ago. If they match, then there is a reasonable confidence the patient is likely still present.

Note that if the patient had actually exited, then only residual heat would remain. Residual heat has been observed to decay substantially over a minute, hence the slow filter's exemplary time constant. If the fast and slow filters match, it is not residual heat that is being monitored since residual heat would have decayed away. In cases where no residual heat exists, exits are very easy to detect. The fast histogram is zero and looks nothing like the slow histogram.

In cases with residual heat, the comparison is more difficult. A modified form of the Kolmogorov-Smirnoff quality of fit (QOF) test may be used. In these residual heat cases, the upper bins of the histograms—the hotter pixel intensities—are where most differences occur. Therefore, the differences in these bins may be scaled relative to the lower bins.

There are some rare cases where the patient exited and residual heat remains such that the fast and slow histograms look very similar. Even the number of pixels can be similar at first. Even so, the residual heat does begin to decay once the patient has left. The normalized fast histogram may continue to resemble the slow histogram, but the number of pixels begins to decrease. Therefore, it may be advisable to also scale the QOF by differences in the total number of pixels in the bins. This accelerates the detection of exits. This fast/slow histogram process and QOF metric may be applied to the nearby frame. Both the inside and nearby QOF metrics are passed to the decision manager.

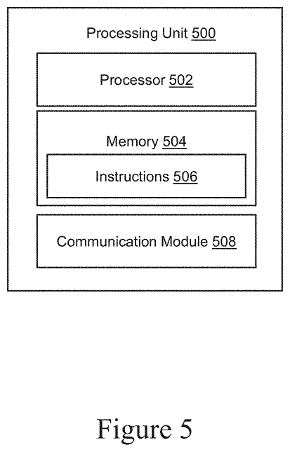

Turning now to FIG. 5, a processing unit 500 is described. The processing unit may be implemented in any of the elements of system 100, including cameras 102, servers 108, and user devices 110, or in processing module 200. As shown, the processing unit 500 may comprise a processor 502, a memory 504 comprising instructions 506, and a communication module 508. These elements may be in direct or indirect communication with each other, for example via one or more buses. The processing unit 500 may be in communication with one or more of the elements of system 100, including cameras 102, servers 108, and user devices 110, or in processing module 200.

The processor 502 include a central processing unit (CPU), a digital signal processor (DSP), an ASIC, a controller, or any combination of general-purpose computing devices, reduced instruction set computing (RISC) devices, application-specific integrated circuits (ASICs), field programmable gate arrays (FPGAs), or other related logic devices, including mechanical and quantum computers. The processor 502 may also comprise another hardware device, a firmware device, or any combination thereof configured to perform the operations described herein. The processor 502 may also be implemented as a combination of computing devices, e.g., a combination of a DSP and a microprocessor, a plurality of microprocessors, one or more microprocessors in conjunction with a DSP core, or any other such configuration.

The memory 504 may include a cache memory (e.g., a cache memory of the processor 502), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an embodiment, the memory 504 includes a non-transitory computer-readable medium. The memory 504 may store instructions 506. The instructions 506 may include instructions that, when executed by the processor 502, cause the processor 502 to perform the operations described herein. Instructions 506 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 508 can include any electronic circuitry and/or logic circuitry to facilitate direct or indirect communication of data between the processing unit 500, and other processors or devices. In that regard, the communication module 508 can be an input/output (I/O) device. The communication module 508 may communicate within the processing unit 500 through numerous methods or protocols. Serial communication protocols may include but are not limited to US SPI, I2C, RS-232, RS-485, CAN, Ethernet, ARINC 429, MODBUS, MIL-STD-1553, or any other suitable method or protocol. Parallel protocols include but are not limited to ISA, ATA, SCSI, PCI, IEEE-488, IEEE-1284, and other suitable protocols. Where appropriate, serial and parallel communications may be bridged by a UART, USART, or other appropriate subsystem.

External communication (including but not limited to software updates, firmware updates, preset sharing between the processor and central server, or readings from the ultrasound device) may be accomplished using any suitable wireless or wired communication technology, such as a cable interface such as a USB, micro USB, Lightning, or FireWire interface, Bluetooth, Wi-Fi, ZigBee, Li-Fi, or cellular data connections such as 2G/GSM, 3G/UMTS, 4G/LTE/WiMax, or 5G. For example, a Bluetooth Low Energy (BLE) radio can be used to establish connectivity with a cloud service, for transmission of data, and for receipt of software patches. The controller may be configured to communicate with a remote server, or a local device such as a laptop, tablet, or handheld device, or may include a display capable of showing status variables and other information. Information may also be transferred on physical media such as a USB flash drive or memory stick.

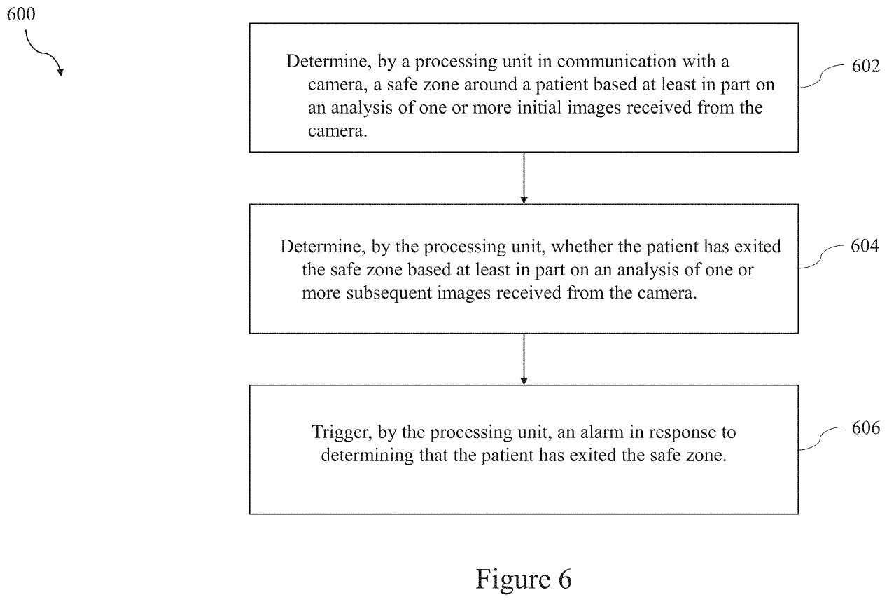

Turning now to FIG. 6, a method is described. The method may be performed by or include any of the elements of system 100, including cameras 102, servers 108, and user devices 110, by processing module 200, or by processing unit 500. The method starts at block 602 where a processing unit, e.g., processing unit 500 or processing module 200, in communication with a camera, e.g., camera 102, determines a safe zone around a patient based at least in part on an analysis of one or more initial images received from the camera. The method continues at block 604 where the processing unit determines whether the patient has exited the safe zone based at least in part on an analysis of one or more subsequent images received from the camera. The method concludes at block 606 where the processing unit triggers an alarm in response to determining that the patient has exited the safe zone.

Further embodiments are contemplated. It is intended that the matter disclosed above and illustrated in the drawings be interpreted as exemplifying particular embodiments and not as limiting the scope of the disclosure.

It is understood that variations may be made in the foregoing without departing from the scope of the present disclosure.

In several embodiments, the elements and teachings of the various embodiments may be combined in whole or in part in some (or all) of the embodiments. In addition, one or more of the elements and teachings of the various embodiments may be omitted, at least in part, and/or combined, at least in part, with one or more of the other elements and teachings of the various embodiments.

Any spatial references, such as, for example, “upper,” “lower,” “above,” “below,” “between,” “bottom,” “vertical,” “horizontal,” “angular,” “upwards,” “downwards,” “side-to-side,” “left-to-right,” “right-to-left,” “top-to-bottom,” “bottom-to-top,” “top,” “bottom,” “bottom-up,” “top-down,” etc., are for the purpose of illustration only and do not limit the specific orientation or location of the structure described above.

In several embodiments, while different steps, processes, and procedures are described as appearing as distinct acts, one or more of the steps, one or more of the processes, and/or one or more of the procedures may also be performed in different orders, simultaneously and/or sequentially. In several embodiments, the steps, processes, and/or procedures may be merged into one or more steps, processes and/or procedures.

In several embodiments, one or more of the operational steps in each embodiment may be omitted. Moreover, in some instances, some features of the present disclosure may be employed without a corresponding use of the other features. Moreover, one or more of the above-described embodiments and/or variations may be combined in whole or in part with any one or more of the other above-described embodiments and/or variations.

Although several embodiments have been described in detail above, the embodiments described are illustrative only and are not limiting, and those skilled in the art will readily appreciate that many other modifications, changes and/or substitutions are possible in the embodiments without materially departing from the novel teachings and advantages of the present disclosure. Accordingly, all such modifications, changes, and/or substitutions are intended to be included within the scope of this disclosure as defined in the following claims. In the claims, any means-plus-function clauses are intended to cover the structures described herein as performing the recited function and not only structural equivalents, but also equivalent structures. Moreover, it is the express intention of the applicant not to invoke 35 U.S.C. § 112(f) for any limitations of any of the claims herein, except for those in which the claim expressly uses the word “means” together with an associated function.

What is claimed is:

1. A patient monitoring system, comprising:
a camera; and
a processing unit in communication with the camera, wherein the processing unit is configured to:
determine, based at least in part on an analysis of one or more initial images received from the camera, a safe zone around the patient,
determine, based at least in part on an analysis of one or more subsequent images received from the camera, whether the patient has exited the safe zone, and
trigger an alarm in response to determining that the patient has exited the safe zone.

2. The system of claim 1, wherein the processing unit is further configured to:
calculate a shift metric based at least in part on patient movement detected in one or more of the subsequent images, and
adjust the safe zone based at least in part on the shift metric.

3. The system of claim 1, wherein determining whether the patient has exited the safe zone comprises determining an N number of safe zone perimeter pixels touched by an image object representative of the patient and determining whether the image object touches greater than N number of pixel layers outside the safe zone.

4. The system of claim 1, wherein the processing unit is further configured to:
calculate a cloak metric based at least in part on a level of patient occlusion detected by the processing unit, and
determine whether the patient has exited the safe zone based at least in part on the cloak metric.

5. The system of claim 1, wherein the processing unit is further configured to calculate a fidget index based at least in part on an amount of patient movement detected in the subsequent images.

6. The system of claim 5, wherein the processing unit is further configured to trigger an alarm when the fidget index exceeds a threshold.

7. The system of claim 1, wherein determining whether the patient has exited the safe zone comprises tracking a silhouette of the patient's head and shoulders.

8. The system of claim 1, wherein the processing unit is configured to determine the safe zone around the patient in response to detecting an enable gesture in one or more of the initial images.

9. A patient monitoring method, comprising:
determining, by a processing unit in communication with a camera, a safe zone around the patient based at least in part on an analysis of one or more initial images received from the camera;
determining, by the processing unit, whether the patient has exited the safe zone based at least in part on an analysis of one or more subsequent images received from the camera; and
triggering, by the processing unit, an alarm in response to determining that the patient has exited the safe zone.

10. The method of claim 9, further comprising:
calculating, by the processing unit, a shift metric based at least in part on patient movement detected in one or more of the subsequent images; and
adjusting, by the processing unit, the safe zone based at least in part on the shift metric.

11. The method of claim 9, wherein determining whether the patient has exited the safe zone comprises determining an N number of safe zone perimeter pixels touched by an image object representative of the patient and determining whether the image object touches greater than N number of pixel layers outside the safe zone.

12. The method of claim 9, further comprising:
calculating, by the processing unit, a cloak metric based at least in part on a level of patient occlusion detected by the processing unit; and
determining, by the processing unit, whether the patient has exited the safe zone based at least in part on the cloak metric.

13. The method of claim 9, further comprising calculating, by the processing unit, a fidget index based at least in part on an amount of patient movement detected in the subsequent images.

14. The method of claim 13, further comprising triggering, by the processing unit, an alarm when the fidget index exceeds a threshold.

15. A patient monitoring system, comprising:
a thermal camera; and
a processing unit in communication with the thermal camera, wherein the processing unit is configured to:
determine, based at least in part on an analysis of one or more initial thermal images received from the thermal camera, a safe zone around the patient,
determine, based at least in part on an analysis of one or more subsequent thermal images received from the thermal camera, whether the patient has exited the safe zone, and
trigger an alarm in response to determining that the patient has exited the safe zone.

16. The system of claim 15, wherein determining whether the patient has exited the safe zone comprises determining whether hot pixels completely fill a column of pixels from top to bottom of a thermal image.

17. The system of claim 15, wherein determining whether the patient has exited the safe zone comprises determining whether one or more pixels have changed temperature in two or more consecutive thermal images received from the thermal camera.

18. The system of claim 15, wherein determining whether the patient has exited the safe zone comprises comparing one or more initial thermal images with one or more subsequent thermal images taken a pre-determined amount of time after the one or more initial thermal images used in the comparison were taken.

19. The system of claim 15, wherein determining the safe zone comprises determining a density score of hot pixels to total area within a boundary.

20. The system of claim 15, wherein determining the safe zone comprises at least one of determining a ratio of height-to-width or determining a distance to an edge of a thermal image.
